(19) United States
(12) Patent Application Publication
(10) Pub. No.: US 2021/0210203 A1
(43) Pub. Date: Jul. 8, 2021

(54) NOTIFICATION ALERT ADJUSTMENT

(71) Applicant: International Business Machines Corporation, Armonk, NY (US)

(72) Inventors: Kulvir Singh Bhogal, Forth Worth, TX (US); Zachary A. Silverstein, Jacksonville, FL (US); Michael Bender, Rye Brook, NY (US); Jeremy R. Fox, Georgetown, TX (US)

(21) Appl. No.: 16/732,375

(22) Filed: Jan. 2, 2020

(51) Int. Cl. G16H 40/67; G16H 10/60; G06N 20/00; G08B 7/06

(52) U.S. Cl. CPC G16H 40/67; G16H 10/60; G16Y 20/40; G08B 7/06; G06N 20/00

(57) ABSTRACT

In an approach to adjusting notification alerts based on biometric data, one or more computer processors receive one or more user preferences from a first user. One or more computer processors receive a prompt from a computing device to generate a notification. One or more computer processors retrieve biometric data associated with the first user. One or more computer processors determine a biometric state of the first user based on the retrieved biometric data. One or more computer processors adjust a notification alert based on the received one or more user preferences and on the determined biometric state of the first user. One or more computer processors generate the notification alert.

### BACKGROUND OF THE INVENTION

The present invention relates generally to the field of Internet of Things, and more particularly to adjusting notification alerts based on biometric data.

The internet of things (IoT) is the internetworking of physical devices (also referred to as “connected devices” and “smart devices”), vehicles, buildings, and other items, embedded with electronics, software, sensors, actuators, and network connectivity that enable these objects to collect and exchange data. The IoT allows objects to be sensed and/or controlled remotely across existing network infrastructure, creating opportunities for more direct integration of the physical world into computer-based systems, and resulting in improved efficiency, accuracy, and economic benefit in addition to reduced human intervention. Each thing is uniquely identifiable through its embedded computing system but is able to interoperate within the existing Internet infrastructure.

Home automation is building automation for a home, called a smart home or smart house. A home automation system may control lighting, climate, entertainment systems, appliances, etc. The home automation system may also include home security such as access control and alarm systems. When connected with the Internet, home devices are an important constituent of the internet of things.

Currently, many industries are trending toward cognitive models enabled by big data platforms and machine learning models. Cognitive models, also referred to as cognitive entities, are designed to remember the past, interact with humans, continuously learn, and continuously refine responses for the future with increasing levels of prediction. Machine learning explores the study and construction of algorithms that can learn from and make predictions on data. Such algorithms operate by building a model from example inputs in order to make data-driven predictions or decisions expressed as outputs, rather than following strictly static program instructions. Within the field of data analytics, machine learning is a method used to devise complex models and algorithms that lend themselves to prediction. These analytical models allow researchers, data scientists, engineers, and analysts to produce reliable, repeatable decisions and results and to uncover hidden insights through learning from historical relationships and trends in the data.

### SUMMARY

Embodiments of the present invention disclose a method, a computer program product, and a system for adjusting notification alerts based on biometric data. The method may include one or more computer processors receiving one or more user preferences from a first user. One or more computer processors receive a prompt from a computing device to generate a notification. One or more computer processors retrieve biometric data associated with the first user. One or more computer processors determine a biometric state of the first user based on the retrieved biometric data. One or more computer processors adjust a notification alert based on the received one or more user preferences and on the determined biometric state of the first user. One or more computer processors generate the notification alert.

### BRIEF DESCRIPTION OF THE DRAWINGS

FIG. 1 is a functional block diagram illustrating a distributed data processing environment, in accordance with an embodiment of the present invention;

FIG. 2 is a flowchart depicting operational steps of a notification adjustment program, on a server computer within the distributed data processing environment of FIG. 1, for adjusting notification alerts in response to biometric data, in accordance with an embodiment of the present invention; and

FIG. 3 depicts a block diagram of components of the server computer executing the notification adjustment program within the distributed data processing environment of FIG. 1, in accordance with an embodiment of the present invention.

### DETAILED DESCRIPTION

The smart device industry is growing exponentially. People are surrounded by a plethora of devices that beep, buzz, ring, and otherwise issue an alert in order to notify a user to an event, whether in the user's home, in another user's home, or in a different environment, such as a hotel room or a vehicle. While many smart devices allow a user to manually toggle the sound associated with the device, the smart devices do not anticipate the state of the user (i.e., the state) and whether or not the user wants to receive the alert based on the state. Embodiments of the present invention recognize that improvement to smart device notification alerts may be gained by using biometric data associated with the user at the time of the notification to determine the user's preferences regarding receiving the notification. Implementation of embodiments of the invention may take a variety of forms, and exemplary implementation details are discussed subsequently with reference to the Figures.

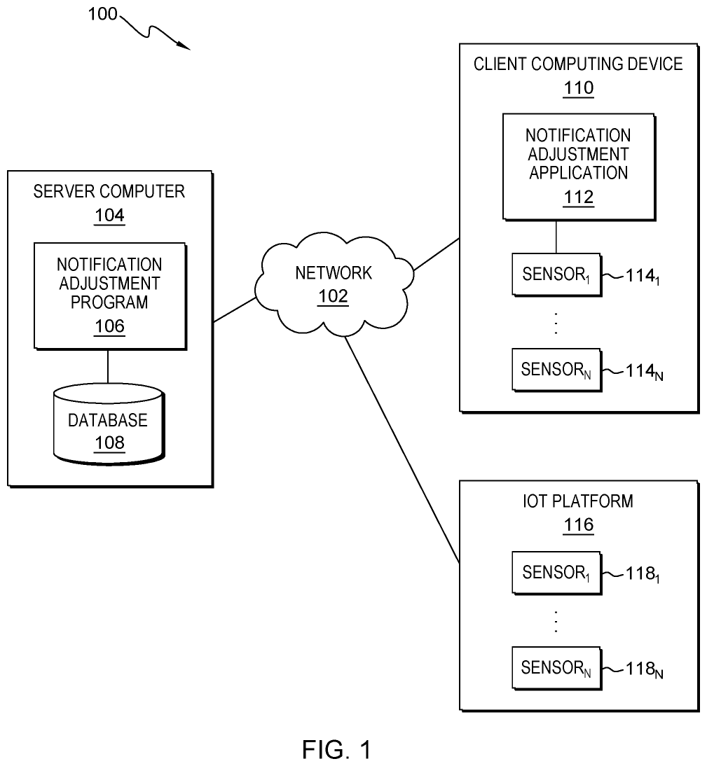

FIG. 1 is a functional block diagram illustrating a distributed data processing environment, generally designated 100, in accordance with one embodiment of the present invention. The term “distributed” as used herein describes a computer system that includes multiple, physically distinct devices that operate together as a single computer system. FIG. 1 provides only an illustration of one implementation and does not imply any limitations with regard to the environments in which different embodiments may be implemented. Many modifications to the depicted environment may be made by those skilled in the art without departing from the scope of the invention as recited by the claims.

Distributed data processing environment 100 includes server computer 104, client computing device 110, and internet of things (IoT) platform 116, all interconnected over network 102. Network 102 can be, for example, a telecommunications network, a local area network (LAN), a wide area network (WAN), such as the Internet, or a combination of the three, and can include wired, wireless, or fiber optic connections. Network 102 can include one or more wired and/or wireless networks capable of receiving and transmitting data, voice, and/or video signals, including multimedia signals that include voice, data, and video information. In general, network 102 can be any combination of connections and protocols that will support communications between server computer 104, client computing device 110, IoT platform 116, and other computing devices (not shown) within distributed data processing environment 100.

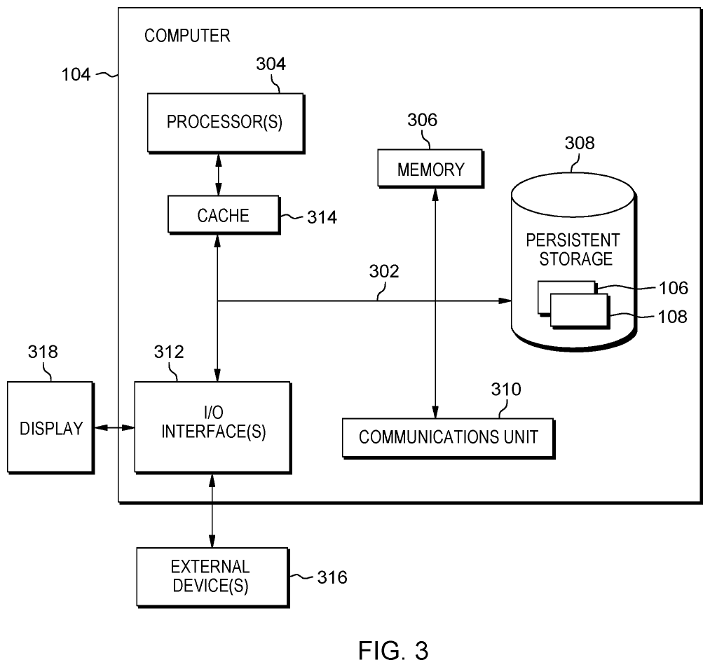

Server computer 104 can be a standalone computing device, a management server, a web server, a mobile computing device, or any other electronic device or computing system capable of receiving, sending, and processing data. In other embodiments, server computer 104 can represent a server computing system utilizing multiple computers as a server system, such as in a cloud computing environment. In another embodiment, server computer 104 can be a laptop computer, a tablet computer, a netbook computer, a personal computer (PC), a desktop computer, a personal digital assistant (PDA), a smart phone, or any programmable electronic device capable of communicating with client computing device 110, IoT platform 116, and other computing devices (not shown) within distributed data processing environment 100 via network 102. In another embodiment, server computer 104 represents a computing system utilizing clustered computers and components (e.g., database server computers, application server computers, etc.) that act as a single pool of seamless resources when accessed within distributed data processing environment 100. Server computer 104 includes notification adjustment program 106 and database 108. Server computer 104 may include internal and external hardware components, as depicted and described in further detail with respect to FIG. 3.

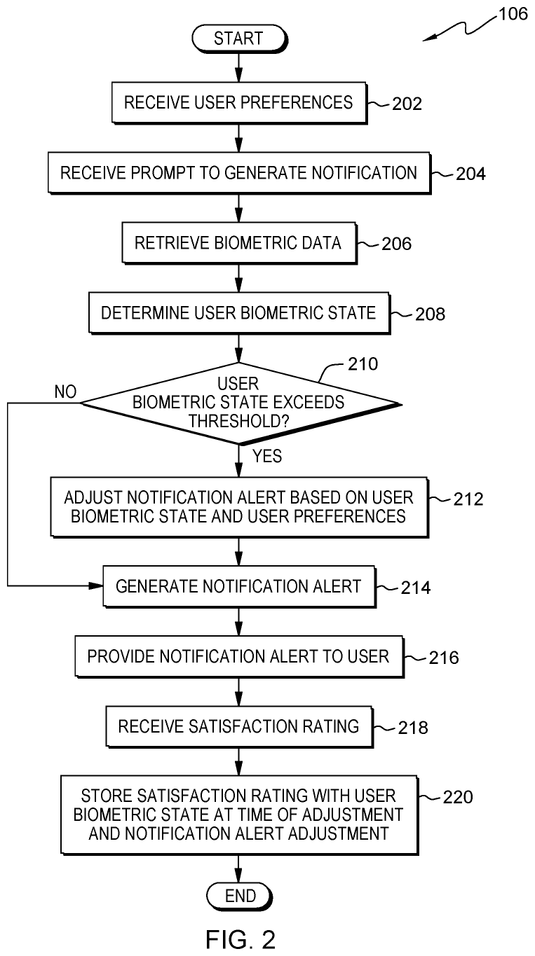

Notification adjustment program 106 determines whether to alert a user to a notification from a smart device based on biometric data associated with the user at the time of the notification. The biometric data indicates a level of notification the user prefers and notification adjustment program 106 adjusts the notification accordingly. Notification adjustment program 106 receives user preferences for a plurality of notifications. Notification adjustment program 106 receives a prompt to generate a notification from an application or another device. Notification adjustment program 106 retrieves biometric data associated with the user, and, based on the retrieved biometric data, notification adjustment program 106 determines the biometric state of the user. Notification adjustment program 106 determines whether the biometric state exceeds a pre-defined threshold. If the biometric state exceeds the pre-defined threshold, then notification adjustment program 106 adjusts the notification alert, based on the user preferences, prior to generating and providing the notification alert to the user. Notification adjustment program 106 receives a satisfaction rating corresponding to the adjustment. Notification adjustment program 106 stores the satisfaction rating with both the corresponding user biometric state at the time of the notification adjustment and the notification alert adjustment. Notification adjustment program 106 is depicted and described in further detail with respect to FIG. 2.

Database 108 is a repository for data used by notification adjustment program 106. Database 108 represents one or more databases. In the depicted embodiment, database 108 resides on server computer 104. In another embodiment, database 108 may reside on client computing device 110 or elsewhere within distributed data processing environment 100, provided notification adjustment program 106 has access to database 108. A database is an organized collection of data. Database 108 can be implemented with any type of storage device capable of storing data and configuration files that can be accessed and utilized by notification adjustment program 106, such as a database server, a hard disk drive, or a flash memory. Database 108 stores user preferences for notification adjustment program 106 to adjust notification alerts. Database 108 also stores biometric data associated with the user. Additionally, database 108 also stores satisfaction ratings. Database 108 also stores notification alert adjustments performed by notification adjustment program 106 as well as the biometric state of the user at the time of the adjustment.

Client computing device 110 can be one or more of a laptop computer, a tablet computer, a smart phone, smart watch, a smart speaker, or any programmable electronic device capable of communicating with various components and devices within distributed data processing environment 100, via network 102. Client computing device 110 may be a wearable computer. Wearable computers are miniature electronic devices that may be worn by the bearer under, with, or on top of clothing, as well as in or connected to glasses, hats, or other accessories. Wearable computers are especially useful for applications that require more complex computational support than merely hardware coded logics. In one embodiment, the wearable computer may be in the form of a head mounted display. The head mounted display may take the form-factor of a pair of glasses. In an embodiment, the wearable computer may be in the form of a smart watch or a smart tattoo. In an embodiment, client computing device 110 may be integrated into a vehicle of the user. For example, client computing device 110 may include a heads up display in the windshield of the vehicle. In general, client computing device 110 represents one or more programmable electronic devices or combination of programmable electronic devices capable of executing machine readable program instructions and communicating with other computing devices (not shown) within distributed data processing environment 100 via a network, such as network 102. Client computing device 110 includes an instance of notification adjustment application 112 and sensor 1141-N.

Notification adjustment application 112 provides an interface between notification adjustment program 106 on server computer 104 and a user of client computing device 110. In one embodiment, notification adjustment application 112 is mobile application software. Mobile application software, or an “app,” is a computer program designed to run on smart phones, tablet computers and other mobile devices. In one embodiment, notification adjustment application 112 may be a graphical user interface (GUI) or a web user interface (WUI) and can display text, documents, web browser windows, user options, application interfaces, and instructions for operation, and include the information (such as graphic, text, and sound) that a program presents to a user and the control sequences the user employs to control the program. Notification adjustment application 112 enables a user of client computing device 110 to provide preferences and satisfaction ratings to notification adjustment program 106 to continually train notification adjustment program 106 to provide notification alert adjustments as desired by the user.

Internet of things (IoT) platform 116 is a suite of components that enable a) deployment of applications that monitor, manage, and control connected devices and sensors; b) remote data collection from connected devices; and c) independent and secure connectivity between devices. The components may include, but are not limited to, a hardware architecture, an operating system, or a runtime library (not shown). In the depicted embodiment, IoT platform 116 includes sensor 1181-N. In another embodiment, IoT platform 116 may include a plurality of other connected computing devices. For example, IoT platform 116 may include home security devices, such as alarms, smoke detectors, and video doorbells. In another example, IoT platform 116 may include a home climate control system or various kitchen appliances. In yet another example, IoT platform 116 may include a virtual assistant. In an embodiment, one or more device included in IoT platform 116 may include a machine learning component which can learn a user's preferences over time by observing the user's actions. For example, an intelligent home climate control system may detect a pattern such as the user setting a thermostat for 65 degrees Fahrenheit in the mornings on Monday through Friday, when the user is not at home, and adjusting the thermostat to 70 degrees Fahrenheit for the rest of the time. Based on this pattern, the IoT device can set the thermostat without user intervention.

A sensor is a device that detects or measures a physical property and then records or otherwise responds to that property, such as vibration, chemicals, radio frequencies, environment, weather, humidity, light, etc. Sensor 1141-N and sensor 1181-N, herein sensor(s) 114 and sensor(s) 118, detect a plurality of attributes of a user of notification adjustment application 112 and of the environment of the user. As used herein, N represents a positive integer, and accordingly the number of scenarios implemented in a given embodiment of the present invention is not limited to those depicted in FIG. 1. Sensor(s) 114 and sensor(s) 118 may be one or more of a plurality of types of camera, including, but not limited to, pin-hole, stereo, omni-directional, non-central, infrared, video, digital, three dimensional, panoramic, filter-based, wide-field, narrow-field, telescopic, microscopic, etc. In some embodiments, sensor(s) 114 and sensor(s) 118 include any device capable of imaging a portion of the electromagnetic spectrum. If client computing device 110 is a wearable device, then sensor(s) 114 may include biometric sensors for detecting the physical condition of the user, such as blood pressure, heart rate, respiratory rate, calories burned, calories consumed, pulse, oxygen levels, blood oxygen level, glucose level, blood pH level, salinity of user perspiration, skin temperature, galvanic skin response, electrocardiography data, body temperature, eye tracking data, etc. Sensor(s) 114 and sensor(s) 118 may be one or more of a plurality of types of microphone for detecting speech and other audible sounds, such as a phone ringing. Sensor(s) 114 and sensor(s) 118 may be one or more of a plurality of types of gyroscopic sensors that can detect movement. Sensor(s) 114 and sensor(s) 118 may be one or more of a plurality of types of pressure sensors. Sensor(s) 114 and sensor(s) 118 may be able to detect weather conditions, such as air temperature, relative humidity, presence and type of precipitation, wind speed, etc., as user preferences may depend on the weather conditions. Sensor(s) 114 and sensor(s) 118 may be global positioning system (GPS) sensors. Sensor(s) 114 and/or sensor(s) 118 may be integrated into the vehicle of the user.

The present invention may contain various accessible data sources, such as database 108, that may include personal data, content, or information the user wishes not to be processed. Personal data includes personally identifying information or sensitive personal information as well as user information, such as tracking or geolocation information. Processing refers to any, automated or unautomated, operation or set of operations such as collection, recording, organization, structuring, storage, adaptation, alteration, retrieval, consultation, use, disclosure by transmission, dissemination, or otherwise making available, combination, restriction, erasure, or destruction performed on personal data. Notification adjustment program 106 and notification adjustment application 112 enable the authorized and secure processing of personal data. Notification adjustment program 106 and notification adjustment application 112 provide informed consent, with notice of the collection of personal data, allowing the user to opt in or opt out of processing personal data. Consent can take several forms. Opt-in consent can impose on the user to take an affirmative action before personal data is processed. Alternatively, opt-out consent can impose on the user to take an affirmative action to prevent the processing of personal data before personal data is processed. Notification adjustment program 106 and notification adjustment application 112 provide information regarding personal data and the nature (e.g., type, scope, purpose, duration, etc.) of the processing. Notification adjustment program 106 and notification adjustment application 112 provide the user with copies of stored personal data. Notification adjustment program 106 and notification adjustment application 112 allow the correction or completion of incorrect or incomplete personal data. Notification adjustment program 106 and notification adjustment application 112 allow the immediate deletion of personal data.

FIG. 2 is a flowchart depicting operational steps of notification adjustment program 106, on server computer 104 within distributed data processing environment 100 of FIG. 1, for adjusting notification alerts in response to biometric data, in accordance with an embodiment of the present invention.

Notification adjustment program 106 receives user preferences (step 202). In an embodiment, notification adjustment program 106 receives user preferences, metadata, and attributes from the user, herein collectively referred to as user preferences, when the user of client computing device 110 inputs preferences via notification adjustment application 112. In another embodiment, notification adjustment program 106 may receive user preferences by learning a user's likes and dislikes with regards to notifications received over time via observations of data received by sensor(s) 114 and/or sensor(s) 118. User preferences for notifications may vary by device from which the notification alert is received, as well as by the user's biometric state when a notification is received. For example, a user may want a loud alert from a smoke detector or carbon monoxide detector, even when the user is sleeping, however the user may not want to receive a push notification from a retail store while the user is sleeping. User preferences for notifications may include, but are not limited to, a time of day to receive a notification, a time of day to prevent receipt of a notification, a type of alert, a level of notification alert, such as volume, brightness, and vibration levels, biometric cues for when to receive or prevent receipt of a notification, as well as situations that include additional users, and biometric data corresponding to a biometric state of the user. For example, a user may prefer to only receive notifications of phone calls from specific work contacts during the user's working hours, and those notifications can be made with a loud tone. In another example, a user may prefer to receive text messages from particular contacts, even while the user is sleeping, but with a moderate volume so as not to startle the user. In a further example, a user may prefer an alarm to be at a loud volume if the user is in deep sleep, but a softer alarm if the user is not in deep sleep, as detected by one or more of sensor(s) 114 and/or sensor(s) 118. In an embodiment, user preferences may also include biometric data indicative of various user states. For example, a particular heart rate, respiration, movement pattern, etc. can be associated with a period when the user is in deep sleep, while a different heart rate, respiration, movement pattern, etc. can be associated with a period when the user is exercising.

Notification adjustment program 106 receives a prompt to generate a notification (step 204). In an embodiment, notification adjustment program 106 receives an input or prompt from either client computing device 110 or a device included in IoT platform 116 indicating that the device is ready to provide a notification to the user at a pre-defined level of alert. In an embodiment, the device ready to provide an alert has to reach a pre-defined threshold prior to prompting notification adjustment program 106. For example, if the device is an oven pre-heating, then the threshold is the set temperature which has to be reached prior to notifying the user that the oven is ready. In another example, if the device is a carbon monoxide detector, then the carbon monoxide level has to reach a pre-defined threshold before notifying the user. Notification adjustment program 106 intercepts the notification such that notification adjustment program 106 can determine whether to or how to provide the notification alert to the user.

Notification adjustment program 106 retrieves biometric data (step 206). In an embodiment where sensor(s) 114 and/or sensor(s) 118 store data in database 108, notification adjustment program 106 retrieves biometric data from database 108. In another embodiment, notification adjustment program 106 may receive biometric data directly from sensor(s) 114 and/or sensor(s) 118. In an embodiment, notification adjustment program 106 retrieves biometric data including, but not limited to, blood pressure, heart rate, respiratory rate, calories burned, calories consumed, pulse, oxygen levels, blood oxygen level, glucose level, blood pH level, salinity of user perspiration, skin temperature, galvanic skin response, electrocardiography data, body temperature, eye tracking data, mobility data, etc. In an embodiment, biometric data may also include images of the user which can indicate the user's state. For example, notification adjustment program 106 may retrieve an image of the user asleep or watching television. In another embodiment, biometric data may also include audio sounds which can indicate the user's state. For example, notification adjustment program 106 may retrieve the sound of the user snoring. In another example, notification adjustment program 106 may detect speech by the user and use one or more natural language processing (NLP) techniques to understand what the user is saying. In another embodiment, biometric data may include the user's location or movement pattern. For example, notification adjustment program 106 may retrieve GPS data associated with the user from sensor(s) 114 and/or sensor(s) 118.

Notification adjustment program 106 determines the user biometric state (step 208). In an embodiment, notification adjustment program 106 determines, based on the retrieved biometric data, the user state corresponding to whether to provide a notification alert. For example, notification adjustment program 106 retrieves biometric data indicating the user has a low heart rate, even breathing, and is lying in bed, not moving, therefore notification adjustment program 106 determines the biometric state of the user is the user is asleep. In another example, notification adjustment program 106 retrieves biometric data indicating the user has a fast heart rate, is sweating, and is moving at a fast pace in the user's neighborhood, therefore notification adjustment program 106 determines the biometric state of the user is the user is going for a daily run.

Notification adjustment program 106 determines whether the user biometric state exceeds a threshold (decision block 210). In an embodiment, notification adjustment program 106 compares biometric data stored as user preferences in database 108 with the currently retrieved biometric data to determine whether one or more biometric data points exceeds a pre-defined threshold. For example, there may be a pre-defined threshold for heart rate, below which is an indication of the user sleeping, while there may be another pre-defined threshold for heart rate, above which is an indication of the user exercising. The pre-defined threshold may be for one type of biometric data, or the pre-defined threshold may be for a combination of two or more types of biometric data. For example, the pre-defined threshold may require that heart rate, respiration, and movement pattern each exceed an individual threshold in order for the biometric state of the user to exceed a threshold.

If notification adjustment program 106 determines the user biometric state exceeds a threshold (“yes” branch, decision block 210), then notification adjustment program 106 adjusts the notification alert based on the user biometric state and on the received user preferences (step 212). In an embodiment, notification adjustment program 106 considers the biometric state of the user and the user preferences corresponding to the biometric state and/or the type of notification and adjusts the notification alert accordingly. In one embodiment, the adjustment may be binary, i.e., either on or off In another embodiment, the adjustment may be within a range of the possible alert, depending on the type of device. For example, a device with an audio alarm may have a range of volume from 0 to 10, or from none to very loud. In another example, a device with a video alarm may have a range from dim to very bright. In a further example, when a text message from a work colleague prompts notification adjustment program 106 to generate a notification, if notification adjustment program 106 determines the user is sleeping based on biometric data and the user has specified in user preferences that the user does not want to be woken up for work-related notifications, then notification adjustment program 106 adjusts the notification alert such that the user does not receive an alert. In another example, if the text message that prompts notification adjustment program 106 to generate a notification is from a family member, notification adjustment program 106 determines the user is sleeping, but the user has specified in user preferences that the user should be gently woken up to receive a family member's text, then notification adjustment program 106 adjusts the notification alert such that, for example, the volume of the alert is lowered to a moderate level or the alert is manifested as a vibration.

In an embodiment, notification adjustment program 106 may consider the presence of more than one user in a location when adjusting a notification alert. For example, if user 1 is sleeping and user 2 is cooking, then notification adjustment program 106 adjusts the volume of an oven timer to be quieter than normal based on user preferences of user 1, which indicate user 1 prefers not to be disturbed, and the fact that user 2 set the timer, enabling the notification alert. Notification adjustment program 106 can be context aware to not disturb user 1 but to alert user 2 by taking a dynamic selection of notification parameters based on the biometric state of both users.

Responsive to adjusting the notification alert, or if notification adjustment program 106 determines the user biometric state does not exceed a threshold (“no” branch, decision block 210), then notification adjustment program 106 generates the notification alert (step 214). In an embodiment where notification adjustment program 106 adjusted the notification alert, notification adjustment program 106 generates an adjusted notification alert. In an embodiment where the user's biometric state did not exceed a threshold, notification adjustment program 106 generates the notification alert as was pre-defined in the prompt.

Notification adjustment program 106 provides the notification alert to the user (step 216). In one embodiment, notification adjustment program 106 provides the notification alert to client computing device 110. In another embodiment, notification adjustment program 106 provides the alert to one or more devices included in IoT platform 116. In an embodiment where user preferences indicate the user does not want to be disturbed, notification adjustment program 106 may wait until the user's biometric data indicates that a notification alert is acceptable to provide the notification alert to the user. In an embodiment, notification adjustment program 106 may determine from which device the user receives the notification. For example, if a user set an alarm on a clock in the user's bedroom, but the user fell asleep on the couch in the living room, then notification adjustment program 106 determines the location of the user prior to providing the notification, and selects a device in close proximity to the user, such as client computing device 110, for the notification such that the user hears the alarm. In an embodiment, in addition to the user's biometric data and preferences, notification adjustment program 106 may detect different priorities for notifications based on historical segmentation and clustering for specific devices. For example, a timer on an oven may have a higher priority alarm than a timer on a microwave because if food is not removed from the oven at the time the timer goes off, then the food may be ruined, versus food in a microwave, which may get cold but would not be burned if left after the timer went off. If a notification from a specific device has a high priority, then notification adjustment program 106 may override user preferences to provide the notification alert.

Notification adjustment program 106 receives a satisfaction rating (step 218). In response to the actions taken by notification adjustment program 106, the user provides a satisfaction rating to notification adjustment program 106. In one embodiment, notification adjustment program 106 receives the satisfaction rating of the notification alert from the user via input by the user into notification adjustment application 112. For example, the user may choose a rating from list of ratings. In another example, the user may enter, via text or speech, a description of the satisfaction rating. In an embodiment, notification adjustment program 106 receives the satisfaction rating of the notification alert from the user in response to a prompt via notification adjustment application 112. In an embodiment, the satisfaction rating may be a numeric scale, for example, one to five. In another embodiment, the satisfaction rating may be a selection of textual terms, for example, very satisfied, satisfied, neutral, dissatisfied, very dissatisfied. In an embodiment, notification adjustment application 112 may ask the user to include a reason for the rating. In another embodiment, notification adjustment program 106 may receive a satisfaction rating by observing the sentiment of the user via sensor(s) 114 and/or sensor(s) 118. For example, notification adjustment program 106 may receive an image or video of the face of the user displaying a smile or a frown. In a further embodiment, notification adjustment program 106 may receive a satisfaction rating via speech or other audible sounds detected by sensor(s) 114 or sensor(s) 118. For example, if a user is startled by the notification, sensor(s) 114 and/or sensor(s) 118 may detect a loud gasp or squeal from the user. In one embodiment, notification adjustment program 106 receives the satisfaction rating immediately upon providing the notification alert. In another embodiment, notification adjustment program 106 may receive the satisfaction rating after a period of time has passed since providing the notification alert.

Notification adjustment program 106 stores the satisfaction rating with the user biometric state at the time of the adjustment and the notification adjustment (step 220). In an embodiment, notification adjustment program 106 creates a matrix of satisfaction ratings versus the biometric state of the user at the time notification adjustment program 106 adjusted the notification alert, and the adjustment made to the notification alert in database 108 along with the user preferences. By storing these data points together, notification adjustment program 106 correlates notification adjustments to user satisfactions. As the process is repeated, notification adjustment program 106 learns preferred notification alert adjustments. In another embodiment, notification adjustment program 106, via machine learning techniques, may also learn preferred notification alert adjustments by determining circumstances, i.e., biometric data and/or user preferences, around the user ignoring a notification alert.

FIG. 3 depicts a block diagram of components of server computer 104 executing notification adjustment program 106 within distributed data processing environment 100 of FIG. 1, in accordance with an embodiment of the present invention. It should be appreciated that FIG. 3 provides only an illustration of one implementation and does not imply any limitations with regard to the environments in which different embodiments can be implemented. Many modifications to the depicted environment can be made.

Server computer 104 can include processor(s) 304, cache 314, memory 306, persistent storage 308, communications unit 310, input/output (I/O) interface(s) 312 and communications fabric 302. Communications fabric 302 provides communications between cache 314, memory 306, persistent storage 308, communications unit 310, and input/output (I/O) interface(s) 312. Communications fabric 302 can be implemented with any architecture designed for passing data and/or control information between processors (such as microprocessors, communications and network processors, etc.), system memory, peripheral devices, and any other hardware components within a system. For example, communications fabric 302 can be implemented with one or more buses.

Memory 306 and persistent storage 308 are computer readable storage media. In this embodiment, memory 306 includes random access memory (RAM). In general, memory 306 can include any suitable volatile or non-volatile computer readable storage media. Cache 314 is a fast memory that enhances the performance of processor(s) 304 by holding recently accessed data, and data near recently accessed data, from memory 306.

Program instructions and data used to practice embodiments of the present invention, e.g., notification adjustment program 106 and database 108, are stored in persistent storage 308 for execution and/or access by one or more of the respective processor(s) 304 of server computer 104 via cache 314. In this embodiment, persistent storage 308 includes a magnetic hard disk drive. Alternatively, or in addition to a magnetic hard disk drive, persistent storage 308 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 308 may also be removable. For example, a removable hard drive may be used for persistent storage 308. Other examples include optical and magnetic disks, thumb drives, and smart cards that are inserted into a drive for transfer onto another computer readable storage medium that is also part of persistent storage 308.

Communications unit 310, in these examples, provides for communications with other data processing systems or devices, including resources of client computing device 110. In these examples, communications unit 310 includes one or more network interface cards. Communications unit 310 may provide communications through the use of either or both physical and wireless communications links. Notification adjustment program 106, database 108, and other programs and data used for implementation of the present invention, may be downloaded to persistent storage 308 of server computer 104 through communications unit 310.

I/O interface(s) 312 allows for input and output of data with other devices that may be connected to server computer 104. For example, I/O interface(s) 312 may provide a connection to external device(s) 316 such as a keyboard, a keypad, a touch screen, a microphone, a digital camera, and/or some other suitable input device. External device(s) 316 can also include portable computer readable storage media such as, for example, thumb drives, portable optical or magnetic disks, and memory cards. Software and data used to practice embodiments of the present invention, e.g., notification adjustment program 106 and database 108 on server computer 104, can be stored on such portable computer readable storage media and can be loaded onto persistent storage 308 via I/O interface(s) 312. I/O interface(s) 312 also connect to a display 318.

Display 318 provides a mechanism to display data to a user and may be, for example, a computer monitor. Display 318 can also function as a touch screen, such as a display of a tablet computer.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product. The computer program product may include a computer readable storage medium (or media) having computer readable program instructions thereon for causing a processor to carry out aspects of the present invention.

The computer readable storage medium can be any tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A method comprising:
receiving, by one or more computer processors, one or more user preferences from a first user;
receiving, by one or more computer processors, a prompt from a computing device to generate a notification;
retrieving, by one or more computer processors, biometric data associated with the first user;
determining, by one or more computer processors, a biometric state of the first user based on the retrieved biometric data;
adjusting, by one or more computer processors, a notification alert based on the received one or more user preferences and on the determined biometric state of the first user; and
generating, by one or more computer processors, the notification alert.

2. The method of claim 1, further comprising receiving, by one or more computer processors, a satisfaction rating from the first user.

3. The method of claim 2, further comprising storing, by one or more computer processors, the received satisfaction rating with the biometric state of the user and an associated adjustment to the notification alert.

4. The method of claim 1, further comprising determining, by one or more computer processors, the biometric state of the user exceeds a pre-defined threshold, wherein the pre-defined threshold is based on the received one or more user preferences.

5. The method of claim 1, wherein the one or more user preferences are selected from the group consisting of: a time of day to receive a notification, a time of day to prevent receipt of a notification, a level of notification alert, a volume of notification alert, a brightness of notification alert, a vibration level of a notification alert, a biometric cue for when to receive a notification, a biometric cue to prevent receipt of a notification, biometric data corresponding to a biometric state of the first user, and a description of a situation that includes an additional user.

6. The method of claim 1, wherein the retrieved biometric data associated with the first user is selected from the group consisting of: blood pressure, heart rate, respiratory rate, calories burned, calories consumed, pulse, oxygen level, blood oxygen level, glucose level, blood pH level, salinity of user perspiration, skin temperature, galvanic skin response, electrocardiography data, body temperature, eye tracking data, and mobility data.

7. The method of claim 1, further comprising:
determining, by one or more computer processors, a second user enabled the computing device to generate a prompt for a second notification; and
providing, by one or more computer processors, a second notification alert to the second user.

8. The method of claim 1, wherein adjusting the notification alert based on the received one or more user preferences and on the determined biometric state of the first user is selected from the group consisting of: a binary adjustment and an adjustment within a possible range of the alert.

9. A computer program product comprising:
one or more computer readable storage media and program instructions collectively stored on the one or more computer readable storage media, the stored program instructions comprising:
program instructions to receive one or more user preferences from a first user;
program instructions to receive a prompt from a computing device to generate a notification;
program instructions to retrieve biometric data associated with the first user;
program instructions to determine a biometric state of the first user based on the retrieved biometric data;
program instructions to adjust a notification alert based on the received one or more user preferences and on the determined biometric state of the first user; and
program instructions to generate the notification alert.

10. The computer program product of claim 9, the stored program instructions further comprising program instructions to receive a satisfaction rating from the first user.

11. The computer program product of claim 10, the stored program instructions further comprising program instructions to store the received satisfaction rating with the biometric state of the user and an associated adjustment to the notification alert.

12. The computer program product of claim 9, the stored program instructions further comprising program instructions to determine the biometric state of the user exceeds a pre-defined threshold, wherein the pre-defined threshold is based on the received one or more user preferences.

13. The computer program product of claim 9, the stored program instructions further comprising:
program instructions to determine a second user enabled the computing device to generate a prompt for a second notification; and
program instructions to provide a second notification alert to the second user.

14. The computer program product of claim 9, wherein the program instructions to adjust the notification alert based on the received one or more user preferences and on the determined biometric state of the first user is selected from the group consisting of: a binary adjustment and an adjustment within a possible range of the alert.

15. A computer system comprising:
one or more computer processors;
one or more computer readable storage media;
program instructions collectively stored on the one or more computer readable storage media for execution by at least one of the one or more computer processors, the stored program instructions comprising:
program instructions to receive one or more user preferences from a first user;
program instructions to receive a prompt from a computing device to generate a notification;
program instructions to retrieve biometric data associated with the first user;
program instructions to determine a biometric state of the first user based on the retrieved biometric data;
program instructions to adjust a notification alert based on the received one or more user preferences and on the determined biometric state of the first user; and
program instructions to generate the notification alert.

16. The computer system of claim 15, the stored program instructions further comprising program instructions to receive a satisfaction rating from the first user.

17. The computer system of claim 16, the stored program instructions further comprising program instructions to store the received satisfaction rating with the biometric state of the user and an associated adjustment to the notification alert.

18. The computer system of claim 15, the stored program instructions further comprising program instructions to determine the biometric state of the user exceeds a pre-defined threshold, wherein the pre-defined threshold is based on the received one or more user preferences.

19. The computer system of claim 15, the stored program instructions further comprising:
program instructions to determine a second user enabled the computing device to generate a prompt for a second notification; and
program instructions to provide a second notification alert to the second user.

20. The computer system of claim 15, wherein the program instructions to adjust the notification alert based on the received one or more user preferences and on the determined biometric state of the first user is selected from the group consisting of: a binary adjustment and an adjustment within a possible range of the alert.
